(19) United States
(12) Patent Application Publication
(10) Pub. No.: US 2009/0062630 A1
(43) Pub. Date: Mar. 5, 2009

(54) Sensor permitting detection of a substance in the body of a living being

(75) Inventors: Arne Hengerer, Erlangen (DE); Robert Krieg, Nurnberg (DE); Sebastian Schmidt, Weisendorf (DE); Ralph Strecker, Erlangen (DE)

Correspondence Address: HARNESS, DICKEY & PIERCE, P.L.C., P.O.BOX 8910, RESTON, VA, 20195, US

(73) Assignee: SIEMENS AKTIENGESELLSCHAFT

(21) Appl. No.: 12/222,776

(22) Filed: Aug. 15, 2008

(30) Foreign Application Priority Data
Aug. 23, 2007 (DE) 10 2007 039 899.0

(51) Int. Cl. A61B 5/145; C12Q 1/00; A61B 5/05; A61F 2/06

(52) U.S. Cl. 600/309; 435/4; 600/410; 623/1.15

(57) ABSTRACT

A sensor is disclosed for permitting detection of a substance in the body of a living being. In at least one embodiment, the sensor includes probe molecules, for binding the substance that is to be detected, and marking elements designed in such a way that the binding of the probe molecules to the substance to be detected is detectable by way of an imaging modality. For example, antigens or pathogens that are present only in a low concentration can be detected.

The present application hereby claims priority under 35 U.S.C. §119 on German patent application number DE 10 2007 039 899.0 filed Aug. 23, 2007, the entire contents of which is hereby incorporated herein by reference.

### FIELD

Embodiments of the present invention generally relate to a sensor permitting detection of a substance in the body of a living being.

### BACKGROUND

In the diagnosis and treatment of tumors, the detection of tumor antigens (tumor markers) is of increasing importance. Tumor antigens are expressed in the body of patients by tumors or metastases and are distributed through the vascular system in the body. By detection of tumor antigens, it is possible, for example after resection of a tumor from a patient's body, to test whether tumor cells are still present, for example in metastases in the patient's body. As a result of their being limited to the vascular system, tumor antigens cannot be detected in biopsy materials.

After resection, radiotherapy or chemotherapy of a tumor, the patient is examined at regular intervals during the tumor therapy. Blood samples are taken and are tested in the laboratory by in vitro diagnostic methods for the presence of tumor markers or tumor antigens. The substantial dilution of the tumor markers in the blood (in the ng/ml range) means that detection is often impossible, even when a measureable concentration of these substances is still present within the affected organ. A further consideration is that said substances are not produced continuously by tumor cells, but only at certain times. This occurs, for example, when some of the tumor cells die. However, it is not possible to ascertain when this is the case. Tumor antigens in the blood are sometimes detectable only for a short time, such that the interval between the follow-up tests is sometimes too great to permit detection. The detection remains negative, even though a further tumor or metastasis is growing in the patient's body.

US 2005/0153379 A1 discloses a probe with which molecules can be detected in vivo in the blood stream of a patient. For this purpose, the probe is introduced into the blood stream and remains there to collect the molecules which, if present in sufficient numbers, are detectable by way of a detector.

### SUMMARY

In at least one embodiment of the present invention, a sensor is made disclosed which permits in vivo detection of tumor markers or tumor antigens in the body.

According to at least one embodiment, a sensor is provided which includes probe molecules for binding a substance that is to be detected, for example a tumor antigen. The sensor further comprises marking elements designed in such a way that the binding of the probe molecules to the substance to be detected is detectable by means of an imaging modality. By way of the sensor it is possible to place, in proximity to a tumor, probe molecules that bind the produced tumor antigen. In the bound state, the marking elements are able to emit a signal for an imaging modality, such as magnetic resonance tomography, fluorescence imaging or computed tomography. Irrespective of the subsequent test and readout of the sensor, the probe molecules are able at any time to bind the substance that is to be detected and thus also to emit a measurement signal at a later time via the marking elements. In at least one embodiment, the sensor includes at least one device for delimiting a reaction volume, said means being designed in such a way that they are open for diffusion of the substance and are impermeable to the probe molecules and the marking elements.

In an example embodiment of the invention, each copy of the probe molecules is designed in such a way that it can bind to several copies of the substance to be detected. Moreover, in an advantageous embodiment, the probe molecule is designed in such a way that several copies of the probe molecules can bind to one copy of the substance to be detected. The agglutination that takes place when the substance to be detected is present facilitates the later detection of the substance by the imaging modality.

In an advantageous embodiment of the invention, a signal for the imaging modality can be generated by the marking elements and can be altered by the binding of the probe molecules to the substance. In this way, the sensor can be read out in the non-activated state without substance to be detected, and a change in the readout signal can be interpreted as the binding of the probe molecules to the substance.

### BRIEF DESCRIPTION OF THE DRAWINGS

Further advantages and configurations of the invention will become evident from the illustrative embodiments explained below with reference to the attached drawings, in which:

FIG. 1 shows an external view of a preferred embodiment of the sensor according to an embodiment of the invention, and

FIGS. 2 and 3 show a schematic representation of the working principle of an example embodiment of the invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

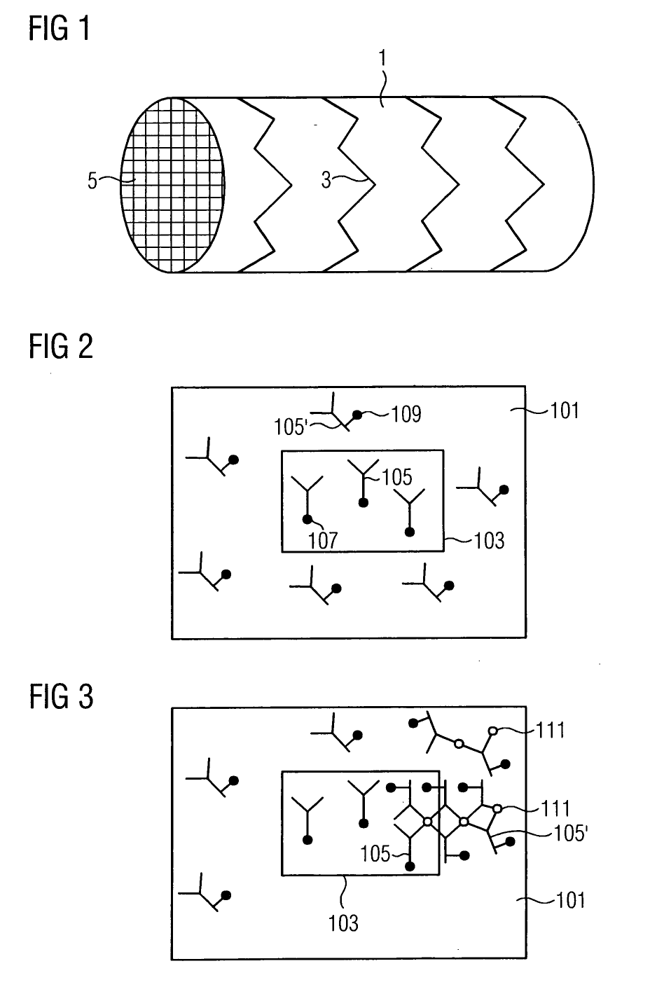

An example embodiment of an implantable sensor according to the invention is shown schematically in FIG. 1. It includes a stent-like lattice tube 1 that can be implanted into a patient's blood stream. The lattice tube 1 can be secured in the patient's blood stream via anchoring units 3. A reaction chamber lying in the inside of the tube, and formed by the latter, is separated from the organism of the patient by semi-permeable membranes 5. The membranes 5 allow blood and its constituents to flow through the reaction chamber of the lattice tube 1 but at the same time ensure that reaction constituents, located in the reaction volume and explained with reference to FIGS. 2 and 3, cannot leave the reaction chamber.

In an alternative embodiment not shown here, the reaction chamber is composed of a three-dimensional polymer net. The latter is configured in such a way that it cannot be degraded by the patient's body. It is made, for example, of non-biodegradable polysaccharides.

The biosensor thus configured with the lattice tube 1 and the internal reaction chamber is implanted, for example during resection of a tumor, in efferent vessels or lymph vessels in spatial proximity to this tumor. In the time following the resection, it is possible that tumor antigens will form from remaining tumor cells or metastases and, over the course of time, will then pass through the reaction chamber of the implanted biosensor.

FIG. 2 shows a schematic plan view of a side wall of the reaction chamber. It is composed of a reaction matrix 101, which comprises an immobilization field 103. Antibodies 105 are immobilized on the immobilization field 103, the antibodies 105 being directed against the tumor antigen to be detected and thus being able to bind to it. The antibodies 105 are connected to the immobilization field 103 via immobilization points 107. These can be effected by covalent bonds, for example. In addition, or alternatively, the antibodies 105 are embedded in a gel matrix composed of a polyalcohol, e.g. sugar or substituted sugar. The antibodies 105 can likewise be embedded in a nano environment of polymeric salts. In this way, they are protected against harmful effects of the blood flowing through, e.g. denaturation or enzymatic degradation.

Further antibodies 105, 105′, also directed against the tumor antigen to be detected, are present in solution within the reaction volume. The antibodies present in solutions are connected, for example, to nanoscale iron oxide particles 109, said particles 109 preferably having a size of between 3 and 250 nm. The connection can be established, for example, by the iron oxide particles having a polymer coating of dextran or starch. This connection will generally be covalent by nature. It is alternately possible to use absorptive or ionic bonds.

The situation in FIG. 2 shows the reaction matrix 101 after implantation. Consequently, no tumor antigen has as yet appeared for detection. By means of the membranes, which limit the reaction volume of the lattice tube 1, the antibodies 105′ present in solution are maintained, with the iron particles 109 bound to them, in the reaction volume. Diffusion through the membrane 5 is not possible.

The pore size of the semipermeable membrane 5 is chosen such that the antibody/particle complex cannot pass through it, but the antigen to be detected can pass through the membrane 5.

The iron oxide particles can be detected, for example by magnetic resonance measurements, within the reaction matrix.

To detect tumor antigens that may possibly have diffused into the reaction matrix, it is necessary to measure these changes by changing the parameters T1, T2 or T2* to be observed.

The situation during and after the presence of tumor antigens is shown in FIG. 3. If tumor antigens 111 diffuse into the reaction matrix 101, they are bound by the antibodies 105 and 105′. The antibodies are configured in such a way that each antibody 105 and 105′ can form a connection with several copies of the tumor antigens 111. The antibodies 105 and 105′ are also configured in such a way that several of the antibodies 105 and 105′ can bind to one tumor antigen 111.

By exploiting this principle, the presence of the tumor antigens 111 leads to agglutination. By way of the immobilized antibodies 105, the clustered structures are held fixedly on the immobilization field 103, which results in a strong concentration of the iron oxide particles 109 near the immobilization field 103. This is associated with a change in the relaxation times, that is to say T2 becomes shorter, and T1 becomes longer as the diameter of the complex increases and as the spatial proximity of the iron oxide particles increases. A more detailed description of one possible method of this kind is found in WO 2002/32291 A2. In this way, it is possible to detect the presence of tumor antigens 111 in the patient's blood stream through the lattice tube 1 by means of MR. The investigation time for the magnetic resonance investigation is independent of the appearance of the tumor antigens 111, as long as it comes later. The measurement signal becomes stronger as the time following the formation of the tumor antigens increases, since the agglutination is able to proceed further. It is thus possible for individual tumor cells that are capable of survival, and that were not noted at the time of resection of a tumor, to be detected via the tumor antigens released.

Instead of using antibodies 105 and 105′, it is also possible to use other suitable ligands, e.g. peptide complexes, anticalcins or antibody derivatives having at least two binding sites for antigens, which are able to bind to the tumor antigens. Suitable ligands can be determined using a histological quick test during the resection of the tumor and can be incorporated into the lattice tube 1. An important aspect here is the long-term stabilization of the iron oxide particles 109 and the antibodies 105 and 105′. These must remain in solution in the absence of the tumor antigen 111 and must not tend to agglutinate or degenerate. Likewise, their shape must remain stable in order to ensure that a connection to the tumor antigens 111 is still possible. The polyalcohol matrices can also be used here. Generally speaking, it is possible to use polyhydroxyl molecules that modify the aqueous environment of the antibodies 105 and 105′ and thus achieve a stabilization. It is also possible to use polyelectrolyes, which further improve the stabilization. Examples of methods for improving the stability of the solution are a PEGylation of the antibodies or a modification with sugars (e.g. dextrans).

In another embodiment, the sensor includes a second reaction chamber, which is closed off from the circulation of blood to the extent that no antigen can penetrate it. This chamber contains the same reagents and is also read out in order to determine an antigen-independent clumping of the reagents over time, e.g. through denaturation processes (negative control). If a partial clumping of the control is established, the test result of the actual sensor is corrected by this effect. If the clumping of the control is too great, such that the signal can no longer be utilized, the sensor has to be replaced.

Through the concentration of the antigen-antibody aggregates in the partial volume of the immobilization field 103, it is possible to establish a quickly and easily detectable signal change in the magnetic resonance signal. Compared to the antibodies 105′, the antibodies 105 can bind, for example, to different epitopes of the tumor antigen 111.

Instead of agglutination, other detection reactions can be used in alternative illustrative embodiments of the invention. Examples of these are Chemical Exchange Saturation Transfer (CEST), as described in EP 1 466 629 A1, and Smart Biosensors (described in U.S. Pat. No. 6,713,045).

Magnetic resonance measurement methods are accordingly available for parametric imaging, e.g. in the product “MapIt” from Siemens.

Other possible illustrative embodiments are optical detection by fluorescence affinity methods, as described in WO 2002/00265 A1, for example, in which the fluorescence signal changes on approach of the molecules.

In computed tomography, clumping of the particles can be presented directly if the resolution of the system exceeds the clump magnitude.

Another possible use of the invention concerns the field of infectious diseases. Here, a similar problem arises if, during treatment, pathogens are present only in very small concentrations in the blood, e.g. in hepatitis C. In this case, conventional methods are often unable to establish whether an active infection exists. The biosensor described here can then be used to collect pathogen-specific molecules over a longer time period and from a larger volume of blood and thus also to reliably detect very small pathogen numbers.

If the presence of the tumor antigens 111 is established in a patient during tests at defined intervals after resection of a tumor, the finding can be confirmed histologically by means of a biopsy. The uncertainties associated with the known methods of in vitro diagnosis are thus eliminated.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A sensor permitting detection of a substance in the body of a living being, comprising:
probe molecules to bind the substance that is to be detected;
marking elements designed in such a way that the binding of the probe molecules to the substance to be detected is detectable by way of an imaging modality; and
means for delimiting a reaction volume, designed in such a way that to be open for diffusion of the substance and to be impermeable to the probe molecules and the marking elements.

2. The sensor as claimed in claim 1, wherein each copy of the probe molecules is designed to bind to several copies of the substance, wherein several copies of the probe molecules are connectable to one copy of the substance.

3. The sensor as claimed in claim 1, wherein a signal for the imaging modality is generateable by the marking elements and alterable by the binding of the probe molecules to the substance.

4. The sensor as claimed in claim 2, wherein agglutination can be caused by the probe molecules in the presence of the substance.

5. The sensor as claimed in claim 1, wherein the sensor is designed to be implantable.

6. The sensor as claimed in claim 1, wherein the marking elements are part of a contrast agent.

7. The sensor as claimed in claim 1, wherein the marking elements comprise at least one magnetic component.

8. The sensor as claimed in claim 7, wherein the magnetic component contains iron oxide.

9. The sensor as claimed in claim 1, wherein the probe molecules are configured as antibodies.

10. The sensor as claimed in claim 9, wherein the antibody binds to a tumor antigen.

11. The sensor as claimed in claim 1, wherein the marking elements are bound to the probe molecules.

12. The sensor as claimed in claim 11, wherein the binding of the marking elements to the probe molecules is achieved by a coating of the marking elements.

13. The sensor as claimed in claim 12, wherein the coating comprises a polymer.

14. The sensor as claimed in claim 1, wherein the sensor comprises a lattice tube for securing in the body.

15. The sensor as claimed in claim 1, wherein the means for delimiting the reaction volume are designed as a semipermeable membrane.

16. The sensor as claimed in claim 1, wherein the means for delimiting the reaction volume are designed as a three-dimensional polymer net.

17. The sensor as claimed in claim 1, wherein the sensor has a surface on which further probe molecules for binding the substance to be detected are immobilized.

18. The sensor as claimed in claim 1, wherein the sensor is designed as a stent.

19. A method for detecting a substance in the body of a patient by way of an implanted sensor as claimed in claim 1, the method comprising:
recording the area of the patient's body containing the sensor by way of an imaging modality;
storing the recorded data in a reference data set;
again recording the area of the body by way of the modality;
storing the recorded additional data in a comparison data set; and
comparing the comparison data set with the reference data set.

20. The method as claimed in claim 19, wherein a movement correction of the data is performed.

21. The method as claimed in claim 19, wherein the imaging modality is magnetic resonance tomography and, on comparing the comparison data set with the reference data set, a change in a relaxation time in the sensor is determined.

22. The sensor as claimed in claim 2, wherein a signal for the imaging modality is generateable by the marking elements and alterable by the binding of the probe molecules to the substance.

23. The method as claimed in claim 20, wherein the imaging modality is magnetic resonance tomography and, on comparing the comparison data set with the reference data set, a change in a relaxation time in the sensor is determined.
